(19) United States
(12) Patent Application Publication
(10) Pub. No.: US 2010/0191344 A1
(43) Pub. Date: Jul. 29, 2010

(54) Set for Producing a Resurfacing Hip Implant

(75) Inventors: Hans Grundei, Lubeck (DE); Ludger Gerdesmeyer, Munich (DE)

Correspondence Address: FLASTER/GREENBERG P.C.;Four Penn Center, 1600 John F. Kennedy Boulevard, 2nd Floor, PHILADELPHIA, PA, 19103, US

(73) Assignee: ESKA IMPLANTS GMBH & CO., KG, Lubeck (DE)

(21) Appl. No.: 12/726,231

(22) Filed: Mar. 17, 2010

Related U.S. Application Data
(63) Continuation of application No. 11/885,569, filed on Sep. 4, 2007, now abandoned.

(30) Foreign Application Priority Data
Mar. 4, 2005 (DE) 10 2005 011 361.3
Jan. 12, 2006 (WO) PTC/EP2006/000212

(51) Int. Cl. A61F 2/32

(52) U.S. Cl. 623/22.11

(57) ABSTRACT

Described here is a set for the construction of a resurfacing hip implant. It comprises a thin metal cup for cemented insertion into the natural acetabulum, a thin metal cap for cemented placement upon the natural femoral head, and an inlay which can be placed in the cup of the acetabulum as a sliding partner for the cap for the femoral head. Also described are methods of using and implanting the implant.

### CROSS REFERENCE TO RELATED APPLICATION

This application claims priority under 35 U.S.C. §120 to, and is a continuation application of previously filed U.S. patent application Ser. No. 11/885,569, filed Sep. 4, 2007, the contents of which are incorporated herein by reference.

### BACKGROUND OF THE INVENTION

Recently, there has been an increase in the use of so called cap implants, which are placed over the readily-prepared residual femoral head, and which can then be fixed in this position. Cap implants consist of a cap modelled on the external form of the natural ball joint, which can be placed on the (part-) prepared residual natural head of the joint. An implant of this type can be constructed from the so-called set for the construction of a reinforcing implant according to DE-C-102 18 801.

To ensure a stable secondary fixation, it is necessary that the bone matter of the residual bone is stable. For this reason, the aforementioned document suggests connecting a pin to the cap for the head of the femur, which is then placed in a corresponding notch in the neck of the femur. This pin has a loosely textured, web-like surface into which and through which the bone trabecula of the surrounding bone material grows, ensuring a stable secondary fixation.

There are however indications which do not require the milling-out of the femoral neck to create room for the pin. An example would be the so-called Legg-Calvé-Perthes disease, which causes aseptic bone necrosis of the emphysis of the femoral head, on one or both sides. This disease occurs primarily in boys aged 4 to 12 (Psychrembel Clinical Dictionary, 259th Edition, 2002, page 1285). Full recovery without deformation is indeed possible, but the risk remains of a cylindrical or mushroom shaped femoral head with flattening of the hip socket, or more rarely, coxa plana or arthrosis deformans.

An example of a further indication would be a cyst in the head of the femur, leading to surface abnormalities of the head of the joint.

In general, necrosis of the head of the joint can lead to surface abnormalities which however still do not justify completely removing the head of the joint, and fitting the patient with a short-stemmed endoprosthesis (EP 0 878 176).

In principle—and recently this has been more strongly acknowledged—it is worthwhile delaying the (part) removal of bone for as long as possible, so that in the event of potential revision surgery at a later date, there are more stages of endoprosthetic fittings to fall back on, from short-stemmed to long-stemmed endoprostheses. The insertion of the latter endoprosthesis requires the complete removal of the femoral neck.

An artificial hip prosthesis became well known from U.S. Pat. No. 4,173,797. But the implantation of this joint requires the use of a form cutter, so the natural bones are affected in the sense that they are significantly freshened, so that blood can flow out of them. Which is why one could not call this a minimally invasive procedure.

DE 94 02 828 U1 takes a first step in the direction of minimally invasive bone removal. It describes an artificial hip joint which consists of a ball-shaped cap and a two-part artificial socket. The cap is used as a replacement joint for the femoral head. For this, a spherical milling of the osseous femoral head is necessary.

Milling-out of the natural acetabulum is also necessary for the insertion of the artificial socket, in the form of a metal cup. So all in all, the implantation of this hip joint still requires that the bone is worked on with a cutter, even if this is to a lesser extent than with the other hip joint which has been referred to. So bone material is always freshened, which leads to bleeding, which, among other things, can have a negative impact on long term fixation.

Against this background, it is the role of the invention in question to present a set for constructing a resurfacing hip implant, mentioned at the outset, which enables the greatest possible flexibility, bearing in mind the long term duration of the implant in situ, i.e. which helps to delay a more extensive endoprosthetic treatment.

### BRIEF SUMMARY OF THE INVENTION

The invention concerns a set for constructing a resurfacing hip implant. This is understood to mean a surface replacement of the natural sliding or articulation surfaces of the acetabulum and the head of the femur.

This task is achieved by a set with the characteristics laid out in claim 1. Advantageous further lessons arise from the subsequent claims.

Accordingly, the set comprises a 1 to 1.5 mm thick metal cup for cemented insertion into the natural acetabulum, which has simply had the cartilage removed and whose outer contours are modelled on the natural acetabulum; as well as a 1 to 1.5 mm thick metal cap for cementing upon the natural, cartilage-cleared femoral head, whose shape is modelled on that of the femoral head; and an inlay which inserts into the cup of the acetabulum, with a material thickness of between 2 to 5 mm, and is a sliding partner for the cap of the femoral head.

Neither the bone of the acetabulum nor the bone of the femoral head is worked on with a cutter, as is the case with the implantation of a total hip replacement. Hip socket and femoral head are simply cleared of cartilage and connective tissue, i.e. ‘de-cartilaged’. Then the cup for the acetabulum, with cement on the back, is placed into the acetabulum, where the cement forms a bond, thus ensuring that the cup sits firmly in the acetabulum. Correspondingly the cap plus cement is pulled over the femoral head, and fixed there by the cement forming a bond. The material thickness of the cup and the cap is between about 1 to 1.5 mm. The material thickness of the inlay is between 2 and 5 mm. This material thickness is seen to balance out the thickness of the cartilage and connective tissue which has been removed. The use of the inlay means that it can be replaced with a new inlay in the event of any abrasion, thus significantly extending the service life of the resurfacing-implant, and thereby delaying a more serious intervention, for example the implantation of a cap implant (DE-C-102 18 801, DE 94 028 28, DE-A-20 39 731) and the necessary bone preparation, were this to become necessary. In contrast to the implants outlined in the documents cited previously, the invented implant is not a replacement implant, rather a reconstructive implant. In the event of a replacement, there are four further stages of endoprosthetic care available, namely firstly the cap implant followed by the short-stemmed prosthesis, according with EP-B-0 878 176. Then at the next stage a meta-diaphyseal endoprosthesis can be deployed, as per the previously unpublished German patent registration 10 2004 051 431. The end of the endoprosthetic provision sees the patient being fitted with one of the well-known long-stemmed endoprostheses. If you work on the basis of an average service life of 15 years for the endoprostheses at each stage, then in theory the insertion of the invented resurfacing hip implant results in a combined prosthetic maintenance period of 75 years. This treatment system therefore offers sufficient spare time for its practical deployment.

The set's inlay is preferably inserted into the cup of the acetabulum in a conical press fit. This is detachable, so that the inlay can be replaced.

If the cap for the femoral head, according to an advantageous further development, has a ceramic surface as the articulation surface, then preferably the inlay will be made of a ceramic composite material, as is for example established in EP-B-0 502 082. This combination of materials has a particularly low level of abrasion.

In the pole region of the cup of the acetabulum, an opening can beneficially be provided, through which a positioning aid can be placed. This positioning aid might for example be a blind screw which, once inserted, can grip onto the base of the socket for an extended period. This performs a guiding function which enables the precise positioning of the cup in the acetabulum.

Particularly preferential would be a further elaboration meaning that, on the inside of the cap for the femoral head, at least two anti-rotation elements protrude into the inside of the cap. This increases the stability of the cap on the femoral head. The anti-rotation elements, once implanted, protrude into the bone of the femoral head, thus preventing any turning of the cap on the femoral head.

In terms of a specific design, the anti-rotation elements are shield shaped. Alternatively they can be pin-shaped.

The operation required to insert the implant is extremely gentle on the patient, as it does not last very long. Experienced surgeons are able to insert the implant in approximately 35 minutes. Only a small incision is needed to do this, which is healed in a matter of days.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown. The invention is explained in greater detail by the illustrated examples. These show:

FIG. 1: the hip implant, assembled from the set, with the cup fixed in the acetabulum and the cap placed on the femoral head

FIG. 2: the individual components of the set

FIG. 3: an internal view of one design of the cap for the femoral head

FIG. 4: an internal view of another design of the cap for the femoral head

### DETAILED DESCRIPTION OF THE INVENTION

FIG. 1 schematically illustrates how the hip implant, which has been constructed from the set, is implanted. Metal cap 2 is placed on the femoral head 6 of the femur 5. It is fixed there with a thin layer of cement on the inside of the cap.

The metal cup 1 is placed in the natural acetabulum 7 and fixed there with a thin layer of cement. The cement used is a very thin liquid.

Inlay 3 is placed in cup 1, which forms the sliding partner for metal cap 2. A positioning aid protrudes into the hip bone, this might for example be a blind screw, which is inserted through an opening in the pole region of cup 1, and protrudes from there into the hip bone.

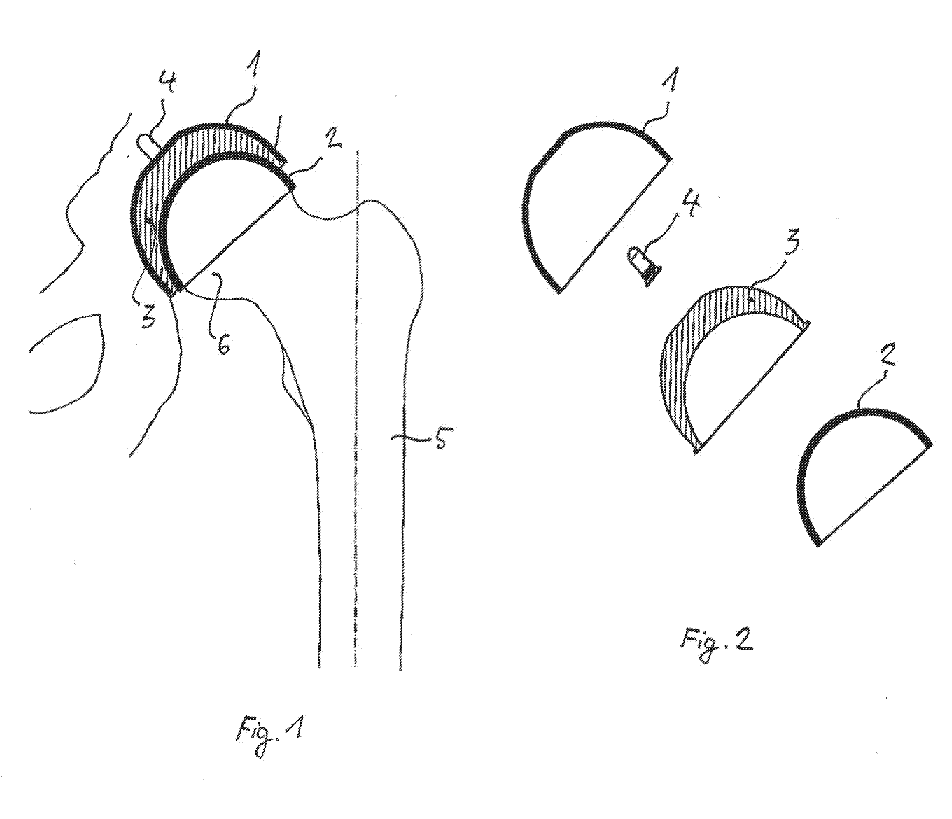

In the construction of the resurfacing hip implant, firstly the positioning aid 4 is placed through the opening in the pole region of cup 1 (FIG. 2). Then comes the insertion of inlay 3 into cup 1. The inlay 3 sits in cup 1 in a (conical) press fit and is replaceable. A specially designated cap 2 is then selected to place upon the femoral head.

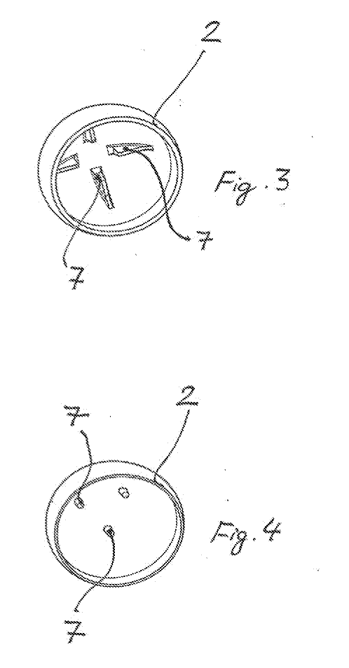

FIG. 3 shows a perspective view of the inside of cap 2 for the femoral head. Clearly visible in the examples shown here are four anti-rotation elements 7 in the shape of shields. Once cap 2 has been placed upon the femoral head, these elements grip onto the osseous material of the femoral head, and ensure that the cap 2 sits on the femoral head without turning. Thus further stabilizing the seating of the cap 2 on the femoral head.

FIG. 4 shows a similar view of the inside of the cap 2, referencing an alternative design. Here, three anti-rotation elements 7 are shaped like pins. These pins also grip onto the osseous material of the femoral head, once the cap had been placed on the head, and thus secure the cap 2 against any rotation on the femoral head.

It will be appreciated by those skilled in the art that changes could be made to the embodiments described above without departing from the broad inventive concept thereof It is understood, therefore, that this invention is not limited to the particular embodiments disclosed, but it is intended to cover modifications within the spirit and scope of the present invention as defined by the appended claims.

1.-16. (canceled)

17. A method of implanting a resurfacing hip implant comprising:
(a) cementing a metal cup into a natural acetabulum, wherein the cup has a thickness of 1 to 1.5 mm;
(b) cementing a metal cap upon a natural femoral head, wherein the cap has a thickness of 1 to 1.5 mm;
(c) inserting an inlay into the cup for the natural acetabulum, wherein the inlay has a material thickness of 2 to 5 mm, and the inlay acts as a sliding partner for the cap for the femoral head,
wherein the natural acetabulum and the natural femoral head have simply had the cartilage removed.

18. The method of claim 17, wherein the inlay is inserted into the cup of the acetabulum in a conical press fit.

19. The method of claim 17, wherein the inlay is formed from a high density polyethylene.

20. The method of claim 17, wherein:
(a) the inlay is formed of a ceramic composite material; and
(b) the cap for the femoral head comprises an articulation surface and the articulation surface is a ceramic surface.

21. The method of 17, wherein the cup for the acetabulum contains an opening located at the pole region of the cup.

22. The method of claim 21, further comprising placing a positioning aid through the opening of the cup for the acetabulum.

23. The method of claim 17, wherein the cap for the femoral head comprises at least two anti-rotational elements that are located on an inside surface of the cap and which protrude into an interior of the cap.

24. The method of claim 23, wherein each of the anti-rotational elements are shield-shaped.

25. The method of claim 23, wherein each of the anti-rotational elements are pin-shaped.
